(19) United States
(12) Patent Application Publication
(10) Pub. No.: US 2026/0103672 A1
(43) Pub. Date: Apr. 16, 2026

(54) SYSTEM, METHOD, AND COMPUTER PROGRAM PRODUCT FOR ASSISTING AN OPERATION OF CELL CULTURE

(71) Applicant: Asahi Kasei Life Science Corporation, Tokyo (JP)

(72) Inventors: Rimi MIYAOKA, Tokyo (JP); Masayoshi MAKI, Tokyo (JP); Manami SUGIMOTO, Tokyo (JP); Hiroki TANIGUCHI, Tokyo (JP); Ippei KIMURA, Tokyo (JP); Ryoichi MATSUI, Tokyo (JP); Takumi AOKI, Tokyo (JP); Daniel BOCK, Fremont, CA (US)

(73) Assignee: Asahi Kasei Life Science Corporation, Tokyo (JP)

(21) Appl. No.: 18/911,517

(22) Filed: Oct. 10, 2024

(51) Int. Cl. C12M 1/36; C12M 1/34

(52) U.S. Cl. CPC C12M 41/48; C12M 41/12; C12M 41/26; C12M 41/30

(57) ABSTRACT

A system, method, and a computer program product for assisting an operation of cell culture are provided. The system comprising a processing circuitry configured to: acquire information relating to the cell culture, calculate predictive values of results of the cell culture over culture time and possible ranges of the respective predictive values, and output the predictive values and the possible ranges of the respective predictive values. The processing circuitry calculates the predictive values at a first point of time, then calculates, on the basis of the predictive values at the first point, the predictive values at a second point of time that is later than the first point of time, and repeats the calculation over the culture time. The processing circuitry uses a statistical regression model to calculate the possible range of the respective predictive values.

### TECHNICAL FIELD

The present disclosure relates to a system, a method, and a computer program product for assisting an operation of cell culture.

### BACKGROUND

In bioproduction, the behavior and productivity of cell cultures vary depending on the cells used and the molecules produced. In recent years, efficient search for optimal culture conditions such as a culture medium, culture method, and a period has been performed using computers.

For example, US 2022/0380717 A1 discloses a method and a device for searching for an optimal culture condition, a cell culture process search method for predicting cell culture results by overviewing the culture process, a culture result prediction program, and a culture result prediction device. A plurality of culture conditions for culturing a cell are generated, and, for each of the plurality of process conditions, a culture prediction result of the cell is acquired. Then, based on the acquired culture prediction result, an optimal process condition is found out.

US 2024/0170098 A1 discloses a method and an apparatus for predicting a cell culture result. A culture environment including a culture medium composition and a culture condition for culturing cells is received, and, on the basis of the culture environment, a biological behavior amount is predicted to change a cell environment on the basis of the predicted biological behavior amount. The prediction of the biological behavior amount and the change of the cell environment are repeated, and the changed cell environment is output.

### SUMMARY

However, it is difficult to accurately predict cell culture results due to biological variability and differences between batches of raw materials. Additionally, since various factors are involved in the cell culture results, it is challenging to identify the relationships between the factors that cause differences in culture outcomes.

It is, therefore, an object of the present disclosure to provide a system, a method, and a computer program product for assisting a cell culture operation that can improve the accuracy of predicting the cell culture results.

In order to achieve the object, one aspect of the present disclosure is a system for assisting an operation of cell culture, the system comprising a processing circuitry configured to:
acquire information relating to the cell culture,
calculate predictive values of results of the cell culture over culture time and possible ranges of the respective predictive values, and
output the predictive values and the possible ranges of the respective predictive values.

As used herein, “cell culture” refers to growth, maintenance, differentiation, transfection, or propagation of cells, tissues, or their products under controlled conditions (e.g., ex vivo).

In one embodiment, the processing circuitry may calculate the predictive values of results of the cell culture at multiple different points of time. In this case, the processing circuitry may calculate the predictive values at a first point of time. Then, based on the predictive values at the first point, the processing circuitry may further calculate the predictive values at a second point of time that is later than the first point of time, and may repeat the calculation over the culture time. The processing circuitry may calculate the predictive value at the first point on the basis of the information relating to the cell culture. Alternatively, the processing circuitry may calculate the predictive value at the first point on the basis of the predictive value at a point of time that is earlier than the first point of time.

The processing circuitry may estimate a change in the predictive values from the first point of time to the second point of time and add the estimated change to the predictive value at the first point to obtain the predictive value at the second point.

The processing circuitry may use a statistical regression model to calculate the possible range of the respective predictive values.

The information relating to the cell culture may include at least one actual value of the results of the cell culture.

The information relating to the cell culture may include a culture condition.

The culture condition may include at least one selected from a group consisting of a type of culture medium, a capacity of a culture apparatus, a volume of initial medium, an amount of cell seeding, a set value of pH, a control range of pH, a lower limit of dissolved oxygen, a set value of temperature, a lower limit of glucose concentration, a gas flow rate, and agitation speed.

The results of the cell culture include at least one selected from a group consisting of cell viability, viable cell density, cell diameter, pH of culture medium, dissolved oxygen level, dissolved carbon dioxide level, a nutrient component, a metabolic component, and an osmotic pressure concentration.

The processing circuitry comprises a trained model obtained by machine learning.

The information relating to the cell culture may include at least one actual value of the results of the cell culture, and the processing circuitry uses the at least one actual value of the results of the cell culture to update the trained model.

The system may further comprise a display unit connected to the processing circuitry and displaying the predictive values and the possible ranges of the respective predictive values.

In one embodiment, the system may further comprise a culture apparatus, and the processing circuitry may be configured to output at least a part of the predictive values and the possible ranges of the respective predictive values to the culture apparatus. Based on the outputs from the processing circuitry, the culture apparatus may control a culture condition. In this case, the culture condition may include at least one selected from a group consisting of a set value of temperature, a gas flow rate, and agitation speed. The culture apparatus may further control the culture condition based on the predictive value and/or the possible range of the predictive value of a nearest future time. Alternatively or additionally, the culture apparatus may terminate the cell culture based on the outputs from the processing circuitry.

In another embodiment, the system may further comprise a culture apparatus and a nutrient composition supplying apparatus. The processing circuitry is configured to output at least a part of the predictive values and the possible ranges of the respective predictive values to the culture apparatus, and the nutrient composition supplying apparatus controls an amount of a nutrient composition supplied to the culture apparatus based on the outputs from the processing circuitry.

Another aspect of the present disclosure is a method for assisting an operation of cell culture, the system comprising:
acquiring information relating to the cell culture,
calculating predictive values of results of the cell culture over culture time and possible ranges of the respective predictive values, and
outputting the predictive values and the possible ranges of the respective predictive values.

In one embodiment, the predictive values of results of the cell culture may be calculated by using a cascade process, and the possible ranges of the respective predictive values are calculated by using a continuous-time stochastic process.

The method may further comprise displaying the predictive values and the possible ranges of the respective predictive values.

Yet another aspect of the present disclosure is a computer program product for assisting an operation of cell culture, the computer program product comprising:
a non-transitory computer-readable medium storing a program which, when executed on a processor, causes the processor to execute operations, the operations comprising:
   acquiring information relating to the cell culture,
   calculating predictive values of results of the cell culture over culture time and possible ranges of the respective predictive values, and
   outputting the predictive values and the possible ranges of the respective predictive values.

The predictive values of results of the cell culture may be calculated by using a cascade process, and the possible ranges of the respective predictive values are calculated by using a continuous-time stochastic process.

The predictive values and the possible ranges of the respective predictive values are calculated by using a trained model obtained by machine learning.

According to the system, method, and computer program product of the present disclosure, it is possible to improve the accuracy of predicting the cell culture results regardless of biological variability and differences between batches of raw materials.

These and other aspects may be understood more readily from the following description and the appended drawings.

### BRIEF DESCRIPTION OF THE DRAWINGS

Various objects, features, and accompanying advantages of the present disclosure beyond those listed above will be fully appreciated when considered in conjunction with the accompanying drawings, in which like reference characters designate the same or similar parts throughout the several views, and wherein:

FIG. 1 is a schematic diagram of a system for assisting an operation of cell culture according to an embodiment of the present disclosure;

FIG. 2 is a block diagram illustrating a schematic configuration of the processing circuitry;

FIG. 3 is a flow diagram illustrating steps implemented by a method for assisting an operation of cell culture according to an embodiment of the present disclosure;

FIG. 4 is a schematic diagram of sequential predictions of the results of the cell culture from day 0 to day n;

FIG. 5 shows an example of the output of the predictive values and the possible ranges of the respective predictive values;

FIG. 6 is a flow diagram illustrating steps implemented by a method for assisting an operation of cell culture according to another embodiment of the present disclosure;

FIG. 7 is a flow diagram illustrating steps implemented by the culture apparatus; and

FIGS. 8A and 8B show feed patterns, predictive values of the antibody titer, and distributions of the predictive values of the antibody titer on day 14 under a bolus feed pattern and a continuous feed pattern, respectively.

### DETAILED DESCRIPTION

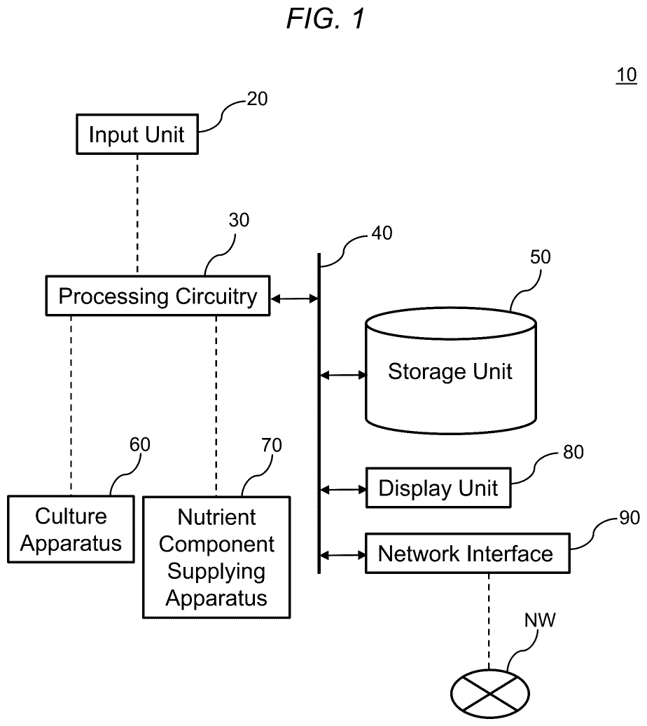

Embodiments will now be described with reference to the accompanying drawings. FIG. 1 is a schematic diagram of a system for assisting an operation of cell culture according to an embodiment of the present disclosure.

A system 10 is designed to assist an operation of cell culture, and can be implemented using a computer. The system includes an input unit 20, a processing circuitry 30, a storage unit 50, a display unit 80, and a network interface 90 connected to a network NW.

The input unit 20 may be, for example, a keyboard, a mouse, a microphone, a digital tablet, a smartphone, or a data logger. In this embodiment, the input unit 20 is separated from and electrically connected to the processing circuitry 30. However, the input unit 20 may be integrated into the processing circuitry 30.

The processing circuitry 30 is connected to a culture apparatus 60 and/or a nutrient component supplying apparatus 70. The culture apparatus 60 includes a culture vessel for culturing cells and a controller configured to control a culture condition, such as a set value of temperature, a gas flow rate, and agitation speed. The nutrient component supplying apparatus 70 includes a feeding unit supplying a nutrient composition, such as glucose or culture medium, to the culture apparatus 60 and a controller configures to control an amount of the nutrient composition supplied to the culture apparatus 60. In this embodiment, the processing circuitry 30 is independent from the culture apparatus 60 and the nutrient component supplying apparatus 70. However, the processing circuitry 30 may be integrated into the culture apparatus 60 and/or the nutrient component supplying apparatus 70.

The storage unit 50 stores any information used for operating the system 10. For example, the storage unit 50 may store a system program, an application program, data from other units, data to be displayed on the display, data acquired from the input unit 20, data received from/transmitted to the network NW, a trained model obtained by machine learning, and so on. The information stored in the storage unit 50 may be updatable by the processing circuitry 30. The storage unit 50 may be, for example, a semiconductor memory, a magnetic memory, or an optical memory. The storage unit 50 is not particularly limited to these, and may include any of long-term storage, short-term storage, volatile, non-volatile, and other memories. Further, the number of memory modules serving as the storage unit 50 and the type of medium on which information is stored are not limited. Alternatively, the storage unit 50 may be integrated into the processing circuitry.

The display unit 80 may include a monitor such as a LCD and is capable of displaying the information input from the input unit 20, information stored in the storage unit 50, the predictive values of the results of the cell culture, a message and/or a warning to be presented to a user, and so on. The user can use the input unit 20 and the display unit 80 to manage the system and method for assisting an operation of cell culture according to the present embodiment.

The network interface 90 communicates with external devices via the network NW. The network NW may be an ad hoc network, a local area network (LAN), a metropolitan area network (MAN), a wireless personal area network (WPAN), a public switched telephone network (PSTN), a terrestrial wireless network, an optical network, or any combination thereof.

In this embodiment, the processing circuitry 30, the storage unit 50, the display unit 80, and the network interface 90 are installed in one location and electrically connected with each other via a bus 40. However, the components may be installed in separate locations and connected with each other via the network NW.

Although the culture apparatus 60 and the nutrient component supplying apparatus 70 are directly connected to the processing circuitry in this embodiment, the culture apparatus 60 and the nutrient component supplying apparatus 70 may be installed in a remote location and communicate with the processing circuitry 30 via the network NW. Further, the culture apparatus 60 and the nutrient component supplying apparatus 70 may be configured separately as shown in FIG. 1 or integrally as a single apparatus.

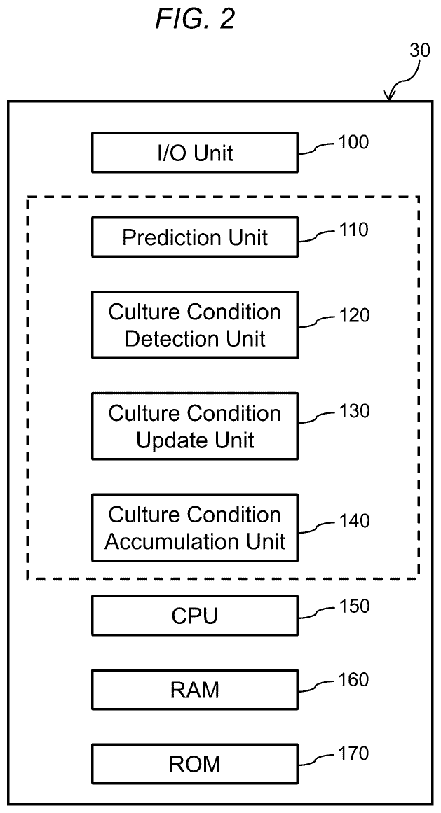

FIG. 2 is a block diagram illustrating a schematic configuration of the processing circuitry 30. The processing circuitry 30 includes an I/O (Input/Output) unit 100, a prediction unit 110, a culture condition detection unit 120, a culture condition update unit 130, a culture condition accumulation unit 140, a CPU (Central Processing Unit) 150, a RAM (Random Access Memory) 160, and a ROM (Read Only Memory) 170.

The I/O unit 100 acquires information relating to the cell culture from the input unit 20.

The prediction unit 110 calculates predictive values of the results of the cell culture and possible ranges of the predictive values of the results of the cell culture by using a trained model obtained by machine learning. The results of the cell culture may be, for example, cell viability, viable cell density, cell diameter, pH of culture medium, dissolved oxygen level, dissolved carbon dioxide level, a nutrient component, a metabolic component, and an osmotic pressure concentration.

The culture condition detection unit 120 compares the predictive values of the results of the cell culture with respective setting upper and/or lower limits and, if at least one of the predictive values of the results of the cell culture is less than the respective setting lower limit and/or more than the respective upper limit, takes a corrective action. The corrective action may be highlighting the predictive values of the results of the cell culture or showing an alert on the display unit 80.

Alternatively, the corrective action may be sending the compared results to the culture condition update unit 130. In this case, the culture condition update unit 130 changes one or more of the culture conditions which is a dominant factor of the growth of cell or cell culture and updates the setting value of the culture conditions. For example, the culture condition update unit 130 generates a command for increasing the dosage of glucose. The predictive values of the results of the cell culture are fed back to the prediction unit 110, and, based on the predictive values of the results of the cell culture, the prediction unit 110 calculates the predictive values of the results of the cell culture on the following date. This cycle is repeated until a predetermined culture period elapsed. In this way, the processing circuitry calculates the predictive values of results of the cell culture at multiple different points in time.

The culture condition accumulation unit 140 accumulates the values of the results of the cell culture and the culture conditions for each day. Alternatively or additionally, the values of the results of the cell culture and the culture conditions for each day may be sent to the storage unit 50 or other unit via the I/O unit 100, such as the display unit 80. A method for assisting an operation of cell culture by using these functions of the processing circuitry 30 will be discussed in detail below. The processing by these functions is performed under the control of the CPU 150.

The processing circuitry 30 discussed above may be a data processing device including, but not limited to, a general-purpose processor that executes software to implement various functions, a programmable logic device (PLD) that is a processor whose circuit configuration can be changed after manufacture, such as an FPGA (Field Programmable Gate Array), and a dedicated electric circuit that is a processor having a circuit configuration designed specifically for executing specific processing, such as an ASIC (Application Specific Integrated Circuit).

The functions of the units discussed above may be implemented by a single processor or a combination of multiple processors. Alternatively, multiple functions may be implemented by a single processor. When the functions of the units discussed above are realized by software, the computer readable codes of the software to be executed are stored in a non-transitory recording medium such as the ROM 170, and the CPU 150 refers to the software. The software to be stored in the ROM 170 includes a program for executing the method for assisting an operation of cell culture, which will be discussed later. Alternatively, the codes may be stored in the storage unit 50 and loaded to the RAM 160 prior to implementing the method.

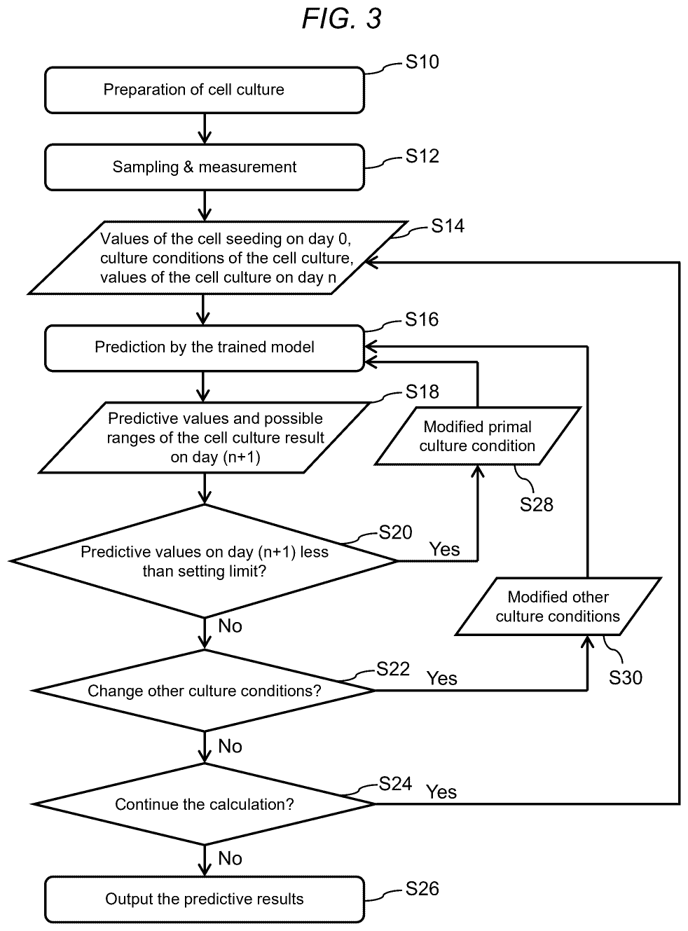

FIG. 3 is a flow chart illustrating steps implemented by a method for assisting an operation of cell culture according to an embodiment of the present disclosure. The method includes steps of acquiring information relating to the cell culture, calculating predictive values of results of the cell culture over culture time and possible ranges of the respective predictive values, and outputting the predictive values and the possible ranges of the respective predictive values.

Prior to a production cell culture, a cell seeding is prepared (S10). At the beginning of the cell culture (day 0), a sample of the cell seeding is taken and subject to measurements of initial information relating to the cell seeding (S12). The measured (actual) information relating to the cell seeding includes biological values of the cell seeding such as, for example, cell viability, viable cell density, cell diameter, pH of culture medium, dissolved oxygen level, dissolved carbon dioxide level, a nutrient component, a metabolic component, and an osmotic pressure concentration.

Then, the cell seeding is inoculated to culture medium in the culture vessel of the culture apparatus 60. The conditions for the production cell culture are input to the system 10 along with the biological values of the cell seeding on day 0 (S14). The culture conditions are, for example, a type of the culture medium, a capacity of the culture apparatus 60 (e.g., an internal volume of the culture vessel), a volume of the culture medium, an amount of the cell seeding, a set value of pH, a control range of pH, a lower limit of dissolved oxygen, a set value of temperature, a lower limit of glucose concentration, a gas flow rate, and agitation speed. Preferably, the culture condition includes at least one of the set values of temperature, a gas flow rate, and agitation speed.

At step S16, the processing circuitry 30 calculates predictive values of results of the cell culture and possible ranges of the respective predictive values on day 1 of the cell culture, which is one day after the beginning of the cell culture, based on the conditions for the production cell culture and the biological values of the cell seeding input at step S14. The calculation may be performed by using a model trained by machine learning. The creation of the trained model by machine learning is not particularly limited, and algorithms such as neural networks, decision trees, support vector machines, or clustering algorithms may be used. These algorithms can be adapted and optimized to enhance the performance and accuracy in the prediction of the results of the cell culture. In this embodiment, the processing circuitry 30 uses a statistical regression model to calculate the predictive values of the cell culture and the possible range of the respective predictive values. Any statistical regression models may be used for the system and method according to the present disclosure, and preferably a Gaussian process regression model is used. Unlike the traditional machine learning models such as a linear regression model and a random forest model, the Gaussian process regression model can not only provide predictions for the target variable but also return the potential variability of the predictions as a standard deviation. The trained model is prepared for each of the results of the cell culture to be predicted. In this embodiment, ten results of the cell culture (i.e., cell viability, viable cell density, cell diameter, pH of culture medium, dissolved oxygen level, dissolved carbon dioxide level, a nutrient component, a metabolic component, and an osmotic pressure concentration) are to be predicted, and thus ten trained models are prepared. Each of the trained models calculates (estimates) a change in the respective result of the cell culture from day 0 to day 1 and adds the change to the input value to obtain the predictive value on day 1.

At step S18, the predictive values of results of the cell culture and possible ranges of the respective predictive values on day 1 are sent to the culture condition detection unit 120, where the predictive values are compared with respective setting upper and/or lower limits. The setting lower and upper limits may be stored in the ROM 170, stored in the storage unit 50 and loaded to the RAM 160, or input with the input unit 20 by the user at the beginning of the cell culture at step S20. If at least one of the predictive values of the results of the cell culture is less than the respective setting lower limit and/or more than the respective upper limit, a predetermined corrective action such as modifying a primal culture condition which is a dominant factor of the growth of cell or cell culture is taken (S28). For example, if the predictive glucose concentration is lower than the setting lower limit of 3.0 g/L, the dosage of glucose is increased by 0.1 ml. The modification of primal culture condition may be automatically done by the processing circuitry 30, or the cell culture is temporarily paused and an alarm notifying the user of the deviation from the setting limits may be popped up on the display unit 80 so that the user may decide the corrective action. The process returns to step S16, and based on the modified primal culture condition, the predictive values of results of the cell culture and possible ranges of the respective predictive values on day 1 of the cell culture are recalculated.

Optionally, at step S22, a necessity for changing any of the culture conditions other than the primal culture condition may be determined. This may also be done automatically manually. If the other condition(s) needs to be changed, the other condition(s) is modified at step S30 and the process returns to step S16. Based on the modified other condition, the predictive values of results of the cell culture and possible ranges of the respective predictive values on day 1 of the cell culture are recalculated.

At step S24, whether or not the calculation is continued is determined. This may also be done automatically manually. If the calculation is decided to be continued, the process returns to step S14 and the predictive values of results of the cell culture on day 1 are used as the input values. At step S16, the processing circuitry 30 calculates the predictive values of results of the cell culture and possible ranges of the respective predictive values on day 2. Specifically, the predictive values of results of the cell culture and possible ranges of the respective predictive value on day 1 (first point of time) are input to the trained models, and the trained models predict differences of the respective results of the cell culture on day 2 (second point of time) from the predictive values of the results on day 1. The calculation is performed in the same manner as for obtaining the predictive values on day 1. Each of the trained models calculates (estimates) a change in the respective result of the cell culture from day 1 to day 2 and adds the change to the predictive value on day 1 to obtain the predictive value on day 2.

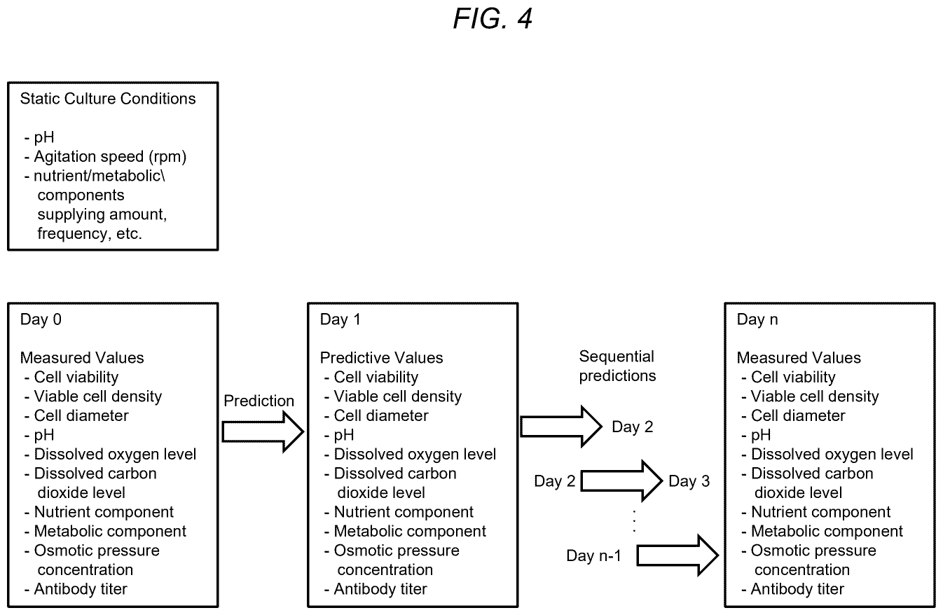

Steps 18, 20, 22, and 24 are performed in the same manner as those discussed above for day 1. This is further repeated for day 3 (third point of time) based on the predictive values on day 2, and day 4 (fourth point of time) based on the predictive values on day 3, and so on until the calculation is decided to be terminated on day n at step S24, as shown in FIG. 4. At step S24, the cell culture is determined to be terminated when a certain condition is satisfied. For example, the calculation is terminated when the predetermined culture period elapses.

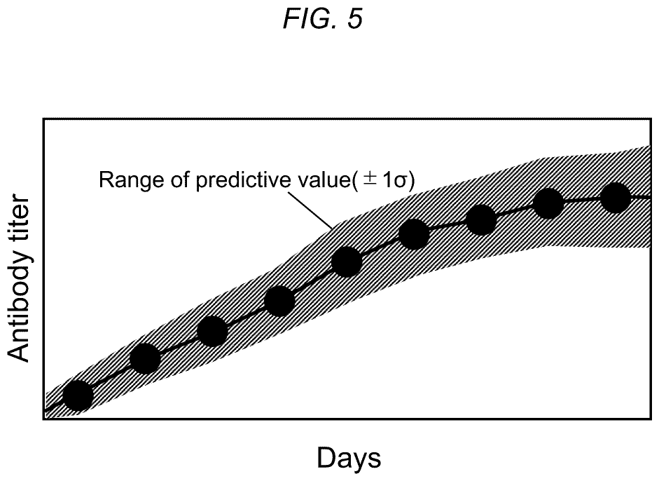

At step S26, the processing circuitry 30 outputs the predictive values and the possible ranges of the respective predictive values thus calculated. For example, the predictive values and the possible ranges of the respective predictive values are sent to the display unit 80 where the predictive values and the possible ranges are displayed in the form of, for example, raw data, a table, or a graph as shown in FIG. 5. In the example shown in FIG. 5, only the antibody tier among the of results of the cell culture is shown. However, two or more results may be displayed. Alternatively or additionally, the predictive values and the possible ranges of the respective predictive values are saved to the storage unit 50 and these results may be used later.

The predictive values and the possible ranges of the respective predictive values may also be output to the nutrient composition supplying apparatus 70, and the nutrient composition supplying apparatus 70 controls the amount of the nutrient composition supplied to the culture apparatus 60 based on the outputs from the processing circuitry 30. Alternatively, the processing circuitry 30 may directly control the nutrient composition supplying apparatus 70 to adjust the amount of the nutrient composition supplied to the culture apparatus 60.

In this embodiment, the calculation is performed at an interval of one day. However, the interval may be longer or shorter, such as a week, a month, a year, 12 hours, an hour, or 30 minutes. Also, the calculation starts at the beginning of the cell culture (day 0). However, the calculation may start at any time during the cell culture (day 1, day 2, . . . ) and the actual information relating to the cell culture measured on a day or days prior to the calculation may be used as the initial information.

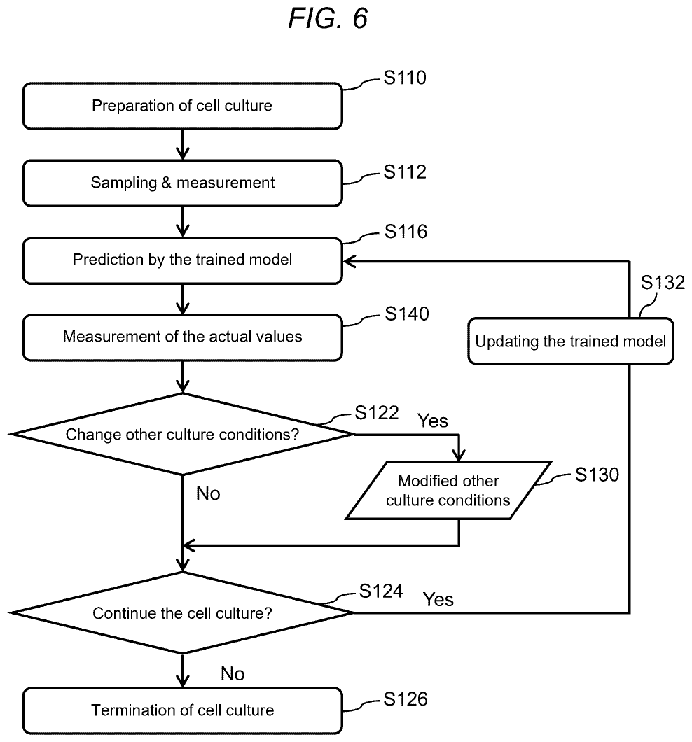

FIG. 6 is a flow diagram illustrating steps implemented by a method for assisting an operation of cell culture according to another embodiment of the present disclosure. In this embodiment, the preparation of cell culture (S110), the sampling and measurement of the cell seeding (S112), and the prediction of the results of the cell culture by the trained model (S116) are performed in the same manner as steps S10-S16 discussed above. The predictive values and the possible ranges of the respective predictive values are calculated until the predetermined culture period elapsed. At least one actual value of the results of the cell culture is measured and input to the system 10 during the cell culture (S140). Optionally, at step S122, a necessity for changing any of the culture conditions other than the primal culture condition may be determined based on the at least one actual value of the results of the cell culture. This may be done automatically manually. If the other condition(s) needs to be changed, the other condition(s) is modified at step S130. For example, if the actual value of the antibody titer deviates from the predictive value of the antibody titer, at least of the set value of temperature the gas flow rate, and the agitation speed may be changed. At step S124, whether or not the cell culture is continued is determined. This may also be done automatically manually. For example, if the actual value of the antibody titer deviates from the predictive value of the antibody titer, the deviation from the predictive value is treated as an indication of an abnormality in the cell culture and the cell culture is determined to be terminated (S126). If the calculation is decided to be continued, the process returns to step S116 and the predictive values of results of the cell culture on day 1 are used as the input values. Then, based on the modified other culture conditions and the at least one actual value of the results of the cell culture, the processing circuitry 30 recalculates the predictive values of results of the cell culture and the possible ranges of the respective predictive values after the day of the measurement. In this way, more accurate predictive values of results at the end of the cell culture can be obtained. The processing circuitry 30 may also use the at least one actual value of the results of the cell culture as the teaching data to update the trained model (S132). The updating of the trained model may be dynamically performed while continuing the cell culture. Alternatively or additionally, the at least one actual value of the results of the cell culture may be measured at one or more points of time during the cell culture, and the at least one actual value of the results of the cell culture may be input into the system 10 as the teaching data to update the trained model after the cell culture is completed.

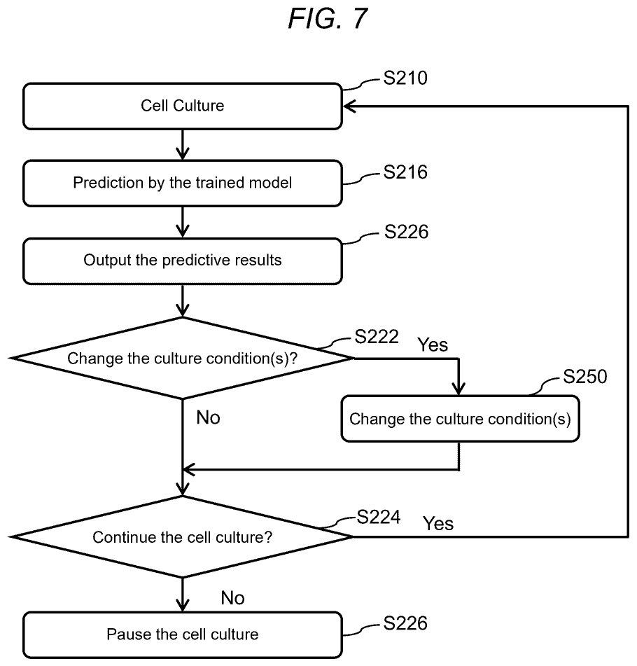

Referring now to FIG. 7, the operation of the culture apparatus will be discussed.

The preparation of cell culture (S210) and the prediction of the results of the cell culture by the trained model (S216) are performed in the same manner as steps S10-S16 discussed above, and the processing circuitry 30 outputs at least a part of the predictive values and the possible ranges of the respective predictive values, which are sent and input to the culture apparatus 60 (S226).

Then, the culture apparatus 60 controls one or more of the culture conditions based on the outputs from the processing circuitry. Specifically, at step S222, a necessity for changing any of the culture conditions is determined. If the culture condition(s) needs to be changed, the culture apparatus 60 changes the culture condition(s) based on the outputs from the processing circuitry 30 at step S250. The culture condition may include a set value of temperature, a gas flow rate, agitation speed, or a combination thereof. The culture apparatus 60 may automatically control the culture condition(s) within the pre-determined optimal range(s) so as to be able to increase the antibody titer at the end of the cell culture. In one embodiment, when the predictive value or the predictive possible range of the viable cell density falls outside the set range or value, the temperature of the culture vessel is controlled within a predetermined range. For example, when the predictive value of the viable cell density is outside the range of 10×10⁶ cells/mL to 200×10⁶ cells/mL, preferably 10×10⁶ cells/mL to 150×10⁶ cells/mL, and more preferably 15×10⁶ cells/mL to 150×10⁶ cells/mL, the culture apparatus 60 controls the temperature of the culture vessel within the range of 30° C. to 35° C., preferably 30° C. to 33° C., and more preferably 30° C. to 32° C. In another embodiment, when the predictive value or the predictive possible range of the carbon dioxide level falls outside the set range or value, the agitation of the culture medium is controlled within a predetermined range. For example, when the predictive value of the carbon dioxide level is outside the range of 5% to 20%, preferably 5% to 15%, and more preferably 5% to 10%, the culture apparatus 60 controls the agitation speed within the range of 101% to 150% of the initial agitation speed, and preferably 101% to 120% of the initial agitation speed. In another embodiment, when the predictive value or the predictive possible range of the internal pressure of the culture vessel falls outside the set range or value, the gas flow rate is controlled within a predetermined range. For example, when the predictive value of the internal pressure of the culture vessel is outside the range of 0.1 psi (0.69 kPa) to 0.6 psi (4.14 kPa), preferably 0.1 psi (0.69 kPa) to 0.5 psi (3.45 kPa), and more preferably 0.2 psi (1.38 kPa) to 0.5 psi (3.45 kPa), the culture apparatus 60 controls the gas flow rate within the range of 50% to 90% of the initial gas flow rate, preferably 50% to 80% of the initial gas flow rate, and more preferably 50% to 70% of the initial gas flow rate. In another embodiment, when the predictive value or the predictive possible range of the carbon dioxide level falls outside the set range or value, the gas flow rate is controlled within a predetermined range. For example, when the predictive value of the carbon dioxide level is outside the range of 5% to 20%, preferably 5% to 15%, and more preferably 5% to 10%, the culture apparatus 60 controls the gas flow rate within the range of 101% to 200% of the initial gas flow rate, and preferably 120% to 180% of the initial gas flow rate.

The predictive value and/or the possible range of the predictive value used by the culture apparatus 60 may be those of a nearest future time. For example, assuming the calculation interval is one day and the cell culture has been performed for three days, the culture apparatus 60 refers to the predictive value and/or the possible range of the predictive value on day 4 (i.e., the nearest future time) to control the culture condition.

Optionally, at step S224, whether or not the cell culture is continued is determined. If the cell culture is decided to be continued, the process returns to step S210. Otherwise, the cell culture is temporarily paused at step S226 so that the user may decide the corrective action. In this way, more ideal results can be obtained at the end of the cell culture.

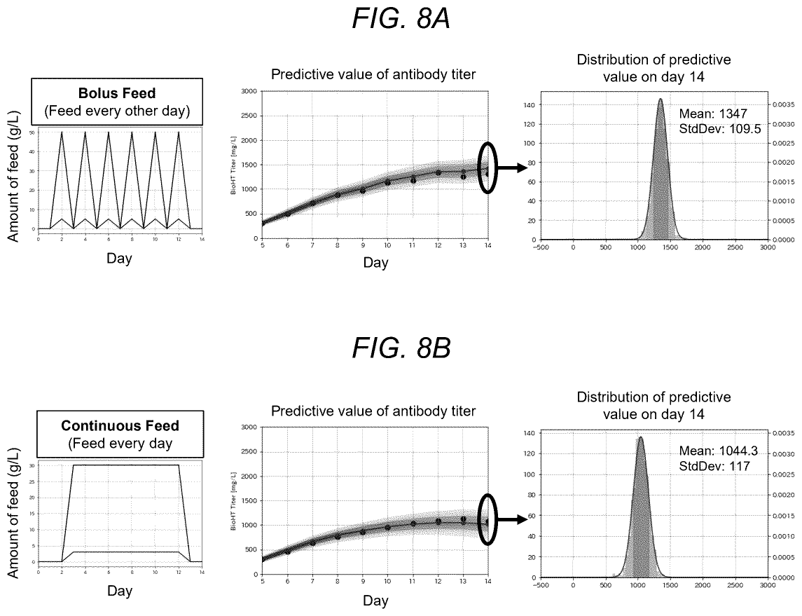

The system and method as discussed above may also be used to simulate the cell culture under different culture conditions. For example, different patterns of feeding the nutrient component may be evaluated. FIGS. 8A and 8B show feed patterns, predictive values of the antibody titer, and distributions of the predictive values of the antibody titer on day 14 under a bolus feed pattern and a continuous feed pattern, respectively. In the simulations, nutrient components A and B are supplied to the culture medium every other day (FIG. 8A) or every day (FIG. 8B), and the predictive values of the antibody titer are calculated by the system 10. The comparison between the distribution of the predictive values on day 14 shows that the bolus feed pattern results in a higher yield with a 97% probability.

According to the above-discussed configuration, the system 10 can provide predictive information that can be used to facilitate decision-making for operators. The system 10 allows the user to input an actual value of the results during the cell culture, so that the accuracy of predicting the cell culture results can be improved regardless of biological variability and differences between batches of raw materials. Moreover, it is possible to evaluate the contribution of each of the culture conditions and formulate the optimal culture conditions. Furthermore, the trained model can be constructed for each culture batch without relying on mathematical models based on existing information.

While the present disclosure has been described with reference to the drawings and examples, it should be noted that various modifications and revisions may be implemented by those skilled in the art based on the present disclosure. Accordingly, such modifications and revisions are included within the scope of the present disclosure. For example, configurations, functions, or the like included in each embodiment can be rearranged without logical inconsistency. In addition, configurations or functions included in each embodiment can be used in combination with another embodiment, and multiple configurations or functions can be combined into one, divided, or partially omitted.

For example, an embodiment in which a general-purpose computer functions as the processing circuitry 30 according to the above embodiments can also be implemented. Specifically, a program in which processes for realizing the functions of the processing circuitry 30 according to the above embodiments are written may be stored in a memory of the general-purpose computer, and the program may be read and executed by a processor of the general-purpose computer. Accordingly, the present disclosure can also be implemented as a program executable by a processor, or a non-transitory computer-readable storage medium storing the program. Examples of the non-transitory computer-readable storage medium include a magnetic storage device, an optical disc, a magneto-optical storage device, and a semiconductor memory. A non-transitory computer-readable storage medium storing a program is also referred to as a computer program product.

For example, in the embodiments described above, the processing circuitry 30 is described to perform all of the operations and processing in the steps shown in FIGS. 3, 6, and 7, the present disclosure is not limited to this configuration. An external server and/or a user terminal may execute some or all of the operations and processing executed by the processing circuitry 30. In such a case, the processing circuitry 30 may be configured to communicate with the external server and/or the user terminal via the network NW.

The actual scope of the protection sought is intended to be defined in the following claims when viewed in their proper perspective based on the prior art.

1. A system for assisting an operation of cell culture, the system comprising a processing circuitry configured to:
acquire information relating to the cell culture,
calculate predictive values of results of the cell culture over culture time and possible ranges of the respective predictive values, and
output the predictive values and the possible ranges of the respective predictive values.

2. The system according to claim 1, wherein the processing circuitry calculates the predictive values of results of the cell culture at multiple different points of time.

3. The system according to claim 2, wherein the processing circuitry calculates the predictive values at a first point of time, then calculates, on the basis of the predictive values at the first point, the predictive values at a second point of time that is later than the first point of time, and repeats the calculation over the culture time.

4. The system according to claim 3, wherein the processing circuitry calculates the predictive value at the first point on the basis of the information relating to the cell culture.

5. The system according to claim 2, wherein the processing circuitry estimates a change in the predictive values from the first point of time to the second point of time and adds the estimated change to the predictive value at the first point to obtain the predictive value at the second point.

6. The system according to claim 1, wherein the processing circuitry uses a statistical regression model to calculate the possible range of the respective predictive values.

7. The system according to claim 1, wherein the information relating to the cell culture includes at least one actual value of the results of the cell culture.

8. The system according to claim 1, wherein the information relating to the cell culture includes a culture condition.

9. The system according to claim 8, wherein the culture condition includes at least one selected from a group consisting of a type of culture medium, a capacity of a culture apparatus, a volume of initial medium, an amount of cell seeding, a set value of pH, a control range of pH, a lower limit of dissolved oxygen, a set value of temperature, a lower limit of glucose concentration, a gas flow rate, and agitation speed.

10. The system according to claim 1, wherein the results of the cell culture include at least one selected from a group consisting of cell viability, viable cell density, cell diameter, pH of culture medium, dissolved oxygen level, dissolved carbon dioxide level, a nutrient component, a metabolic component, and an osmotic pressure concentration.

11. The system according to claim 1, wherein the processing circuitry comprises a trained model obtained by machine learning.

12. The system according to claim 11, wherein the information relating to the cell culture includes at least one actual value of the results of the cell culture, and the processing circuitry uses the at least one actual value of the results of the cell culture to update the trained model.

13. The system according to claim 1, further comprising a display unit connected to the processing circuitry and displaying the predictive values and the possible ranges of the respective predictive values.

14. The system according to claim 1, further comprising a culture apparatus,
wherein the processing circuitry is configured to output at least a part of the predictive values and the possible ranges of the respective predictive values to the culture apparatus, and
the culture apparatus controls a culture condition based on the outputs from the processing circuitry.

15. The system according to claim 14, wherein the culture condition includes at least one selected from a group consisting of a set value of temperature, a gas flow rate, and agitation speed.

16. The system according to claim 14, wherein the culture apparatus controls the culture condition based on the predictive value and/or the possible range of the predictive value of a nearest future time.

17. The system according to claim 1, further comprising a culture apparatus,
wherein the processing circuitry is configured to output at least a part of the predictive values and the possible ranges of the respective predictive values to the culture apparatus, and
the culture apparatus terminates the cell culture based on the outputs from the processing circuitry.

18. The system according to claim 1, further comprising a culture apparatus and a nutrient composition supplying apparatus,
wherein the processing circuitry is configured to output at least a part of the predictive values and the possible ranges of the respective predictive values to the culture apparatus, and
the nutrient composition supplying apparatus controls an amount of a nutrient composition supplied to the culture apparatus based on the outputs from the processing circuitry.

19. A method for assisting an operation of cell culture, the system comprising:
acquiring information relating to the cell culture,
calculating predictive values of results of the cell culture over culture time and possible ranges of the respective predictive values, and
outputting the predictive values and the possible ranges of the respective predictive values.

20. A computer program product for assisting an operation of cell culture, the computer program product comprising:
a non-transitory computer-readable medium storing a program which, when executed on a processor, causes the processor to execute operations, the operations comprising:
acquiring information relating to the cell culture,
calculating predictive values of results of the cell culture over culture time and possible ranges of the respective predictive values, and
outputting the predictive values and the possible ranges of the respective predictive values.
